(19) United States
(12) Patent Application Publication
(10) Pub. No.: US 2013/0052271 A1
(43) Pub. Date: Feb. 28, 2013

(54) Compositions and Methods for Treating Pain

(71) Applicant: Richard S. Sternasty, Connersville, IN (US)

(72) Inventor: Richard S. Sternasty, Connersville, IN (US)

(21) Appl. No.: 13/219,923

(22) Filed: Aug. 29, 2011

(51) Int. Cl. A61K 35/60; A61P 43/00; A61P 19/02; A61P 25/00; A61K 31/7034; A61P 29/00

(52) U.S. Cl. 424/523; 514/35

(57) ABSTRACT

The present invention provides compositions and methods for treating pain in mammals. A composition of the invention comprises one or more anti-inflammatory agents and optionally contains one or more anti-oxidants and immune system boosting agents.

### FIELD OF INVENTION

The invention relates to the fields of pharmacology and medicine, and provides therapeutic methods and compositions for treating pain including chronic pain and pain associated with musculoskeletal diseases such as arthritis and rheumatism.

### DESCRIPTION OF RELATED ART

Pain of various types is a leading cause of discomfort and disability in the United States today. Pain associated with the musculoskeletal system can arise from multiple causes including over-exertion, trauma, and disease. For example, pain from diseases such as arthritis are increasingly problematic as the average age of the population rises. Left untreated, arthritic pain can severely compromise an affected individual's quality of life. According to recent statistics from the Centers for Disease Control and Prevention (CDC), arthritis and other rheumatic conditions account for about 744,000hospitalizations and 4 million days of care in the United States on an annual basis. Forty million Americans, representing 15% of the population, have some form of arthritis, and that figure is expected to increase to 59.4 million (18.2%) by the year 2020. Arthritis costs the nation $65 billion annually in medical costs and lost productivity, and these figures are expected to rise.

Arthritis can manifest in many different forms. Osteoarthritis (OA), or degenerative joint disease, is the most common type. In 1990, OA affected about 20.7 million people. R. C. Lawrence et al. Arthritis & Rheumatism, 41, 778-799, 1998. Osteoarthritis usually presents as joint pain that worsens with exercise. Common joints affected by OA are the knees, hips, spine, shoulders, fingers, and toes. Osteoarthritis is characterized by degeneration of articular cartilage. By age 60, almost all Americans experience at least mild osteoarthritis pain in their neck or spine.

Perhaps an even more devastating form of arthritis is rheumatoid arthritis (RA) which affects approximately 1-2% of the population. RA is caused by an overproduction of pro-inflammatory cytokines including, for example, tumor necrosis factor alpha (TNFα), interleukin 1 (IL-1), coupled with a lack of anti-inflammatory cytokines, including IL-10 and IL-11. RA is characterized by severe pain and synovial inflammation, which progresses to cartilage destruction, bone erosion, and subsequent joint deformity.

It is now believed that free radicals may have a causative affect in the onset and/or progression of arthritic conditions. Bucci L. Healing Arthritis the Natural Way Arlington, Tex., Summit Publishing Group, 1995, pp. 34-5. Free radicals are byproducts of normal metabolism and are pervasive in the environment. Environmental sources for free radicals include tobacco smoke, pollutants, car exhaust, bacteria, radiation, and chemicals, all of which are capable of oxidizing or damaging otherwise healthy cells and leading to a wide variety of pathologies.

Current treatments for pain associated with arthritis and other conditions commonly focus on reducing inflammation while relieving pain. Non-steroidal anti-inflammatory drugs (NSAIDs) are commonly prescribed as a first line of treatment for arthritis and other forms of musculoskeletal pain. However, long-term use of NSAIDs can lead to undesirable consequences including gastric ulcers, kidney damage, hearing loss and inhibition of cartilage formation. In addition, many people are sensitive to aspirin and/or NSAIDs and cannot take them for pain relief due to ancillary medical conditions. While other classes of pain reliever are known, including both narcotic and non-narcotic agents, use of such drugs may in some instances be ineffective, or carry a risk of abuse or tolerance.

Alternative medicine holds out promise for new treatments for pain associated with arthritis and other musculoskeletal diseases and conditions. Plants and herbs have long been used for medicinal purposes. For example, aspirin was originally derived from the bark of a white willow tree, and digitalis from a flower commonly known as fox glove.

While many natural and synthetic agents exist for alleviating pain, there remains a need for improved treatments to safely and effectively reduce chronic and/or acute pain associated with over-exertion, trauma, post-surgery recovery, and/or neurological, neuromuscular, and/or musculoskeletal disease.

### BRIEF SUMMARY OF THE INVENTION

The present invention relates to compositions and methods for reducing pain in a mammal, including a human. Certain variations of the present invention provide improved treatment for pain, including but not limited to, pan due to exercise, injury, trauma, post-surgery recovery, or pain associated with a musculoskeletal disease or condition including, for example, joint, muscle, tendon and/or ligament pain, trauma, or over-exertion, bone pain, fibromyalgia, arthritic pain, joint inflammation, autoimmune disease, and vasculitic disorders including systemic lupus erythematosus, polymyalgia rheumatica, and polyarteritis nodosa.

The invention provides methods for treating and/or reducing pain comprising administering an effective amount of a composition comprising one or more anti-inflammatory agent(s) preferably derived from a natural and/or herbal source.

The invention further provides pharmaceutical compositions for the treatment of pain which comprise one or more anti-inflammatory agent(s) and optionally including one or more other agent(s) including anti-oxidant agent(s).

The invention also provides methods for producing pharmaceutical compositions and articles of manufacture for the treatment of pain.

This Summary is provided to introduce certain concepts, and is not intended to identify any key or essential features of the claimed subject matter.

### DETAILED DESCRIPTION

As used herein the term “pain” refers to any form of chronic or acute pain including, but not limited to, neurological, neuromuscular, and musculoskeletal pain or discomfort experienced by a mammal, including a human, associated with, for example, trauma, injury, strain, sprain, over-exertion, post-surgery recovery, and diseases including, but not limited to, arthritis, osteoarthritis, degenerative arthritis, rheumatoid arthritis, rheumatism, gout, fibromyalgia, lupus, neuropathy, sciatica, joint pain, and back pain, affecting any part of the body.

As used herein, the term “topical administration” or “topical application” refers to application or administration of a composition of the invention to the skin or mucosa, for example, by rubbing a cream, ointment, gel, balm, salve, or the like, directly or indirectly on the surface of the skin to relieve pain. Topical application may also include administration by means of bandages or patches, and the like, in which a composition of the invention is impregnated in or on such devices including transdermal patch devices for transfer to and through the skin.

As used herein, the terms “compound”, “agent”, and “ingredient” will generally, but not necessarily, refer to pharmaceutically active ingredient(s) in a composition of the invention. Preferably, the active ingredient(s) of a composition of the invention are derived from natural, botanical, or herbal sources, including agents and/or products sold commercially as nutraceuticals in health food stores. Thus, in describing an embodiment of a composition as containing, for example, one or more anti-oxidants, or one or more other compounds, agents, or ingredients having anti-oxidant activity, it is to be understood to mean either purified agents and/or compounds, preferably derived from a natural or herbal source having such activity, or to unpurified compounds or products that contain one or more such agents, e.g. anti-oxidant agent(s).

As used herein “subject” will generally refer to a human patient but may also include other mammals such as, but not limited to, horses, cows, sheep, pigs, dogs, cats, and primates.

### Pharmaceutical Compositions

As used herein, the term “pharmaceutical composition” refers to a liquid, semi-solid, or solid composition that contains one or more pharmaceutically active ingredient(s) (e.g., anti-inflammatory agent(s)) and, optionally, other agents including one or more antioxidant(s) and/or immune system boosting agent(s) and/or other carrier, diluent, thickener, surfactant, or other pharmaceutically acceptable excipient.

Pharmaceutical compositions according to the present invention may take any physical form which is pharmaceutically acceptable for topical or oral administration. Pharmaceutical compositions for topical administration are particularly preferred.

In one embodiment of the present invention, a pharmaceutical composition comprises an effective amount of one of more anti-inflammatory agent(s). The anti-inflammatory agent(s) provides pain relief from symptoms, and/or conditions such as, for example, inflammation, stiffness, swelling, redness, and fever. Suitable anti-inflammatory agents include one or more natural product compound(s) selected from the group consisting of plant-based oils, aloe vera, MSM, emu oil, chondroitin, glucosamine, a capsaicinoid, arnica extract, grape seed extract, coriander oil, marigold extract, nettle leaf extract, Roman chamomile oil, blue-bottle extract, St. John's wort, White willow bark extract, witch hazel extract, feverfew extract, barley grass, black cohosh, black snakeroot, bugbane, squawroot, Boswellia, borage oil, bromelain, burdock, calendula, cayenne, dandelion, devil's claw root, DHEA (dehydroepiandosterone), Echinacea, EFAs (essential fatty acids such as omega-3 and omega-6 fatty acids including linoleic acid (LA) and alpha-linolenic acid (LNA)), fish oil (source for EPA and DHA), green tea, pomegranate extract, ginger root, hyssop, elderflower, evening primrose oil, flaxseed, ginkgo, ginger, ginseng, Hawthorne, kaempferol, licorice, life root, golden Senecio, squaw weed, golden groundsel, cocash weed, coughweed, ragwort, golden ragwort, linden, marjoram, meadow sweet, NDGA, neem, neem oil, Padma 28, quercetin, turmeric, wild yam, wormwood, yucca, and combinations thereof.

Anti-inflammatory agents according to the present invention may also include one or more commonly known non-steroidal anti-inflammatory drugs (NSAIDs), such as for example aspirin, diclofenac, ibuprofen, and COX-2 inhibitors, such as celecoxib and rofecoxib. A suitable anti-inflammatory agent may also include steroids such as hydrocortisone, prednisone, triamcilone, betamethasone, and dexamthasone.

The one or more anti-inflammatory agents generally comprise greater than about 50% to greater than about 80% of a composition according to the invention. Preferably, the anti-inflammatory component comprises greater than about 70% of the composition. The one or more anti-inflammatory agents is believed to provide a synergistic benefit when combined with other agents according to the present invention.

In another embodiment, a composition of the present invention also includes an effective amount of at least one anti-oxidant agent(s). Without intending to be bound by any particular theory, it is believed that an anti-oxidant agent(s) may help prevent or reduce tissue damage that may be associated with or accompany pain. Suitable anti-oxidants include chondroitin, vitamin C, grape seed extract, St John's wort extract, coriander oil, barley grass, bilberry, Echinacea, garlic, ginger, ginkgo, ginseng, grape seed, proanthocyanidin extract (GSPE), green tea, Hawthorne, lemon balm, milk thistle, oregano, peppermint, pomegranate juice, purslane, pycnogenol, red wine, rosemary, schizandra, wuweizi, wurenchun, trilinolein, sanchi, turmeric and combinations thereof. Suitable anti-oxidants also include d-alpha Tocopherol, β-carotene, selenium, and vitamin C ester. A particularly preferred anti-oxidant is pynogenol.

The one or more anti-oxidant agent(s) generally comprise about 0.5% to about 10% of the composition; preferably from about 1% to about 5% of the composition.

In another embodiment, compositions of the present invention may also include an effective amount of one or more immune system boosting agent(s) including, for example, echinacea, goldenseal astragalus, garlic, elderberry, and Cat's Claw.

When included, immune system boosting agent generally comprises from about 5% to about 20% of the composition; preferably from about 10% to about 15% of the composition.

In another embodiment, a composition of the present invention may also include a humectant. The humectant can comprise at least one compound selected from the group consisting of sorbitol, ethylene glycol, diethylene glycol, triethylene glycol, and other polyethylene glycols, propylene glycol, dipropylene glycol and other propylene glycols, 1,3-butylene glycol, 1,4-butylene glycol and other butylene glycols, glycerol, diglycerol and other polyglycerols, mannitol, xylitol, maltitol and other sugar alcohols, glycerol ethylene oxide (EO) and propylene-oxide (PO) adducts, sugar alcohol EO and PO adducts, adducts of EO or PO and monosaccharides such as galactose and fructose, adducts of EO or PO and polysaccharides such as maltose and lactose, sodium pyrrolidonecarboxylate, and polyoxyethylene methyl glycoside.

A humectant can comprise from about 0,05% to about 1 % of the composition. Preferably, a humectant comprises from about 0.1 % to about 0.7% of the composition. An optimum concentration of humectant is about 0.7% of the composition.

In another embodiment, the composition also includes a preservative. Suitable preservatives include but are not limited to at least one agent selected from the group consisting of grape seed extract, methylparaben, propylparaben, diazolindinyl urea, and combinations thereof.

If included, a preservative comprises from about 0.05% to about 0.20% of the composition. Preferably, a preservative comprises from about 0.07% to about 0.15% of the composition. An optimum concentration of preservative is about 0.1% of the composition.

In another embodiment, the composition optionally may also include a thickener such as, but not limited to, Carbomer-940 for purposes of producing a composition suitably formulated for topical administration. Carbomer-940 can comprise from about 0.05% to about 15% of the composition. Preferably, a thickener comprises from about 0.07% to about 10% of the composition. An optimum concentration of thickener is about 0.1% of the composition.

Other non-active ingredients may optionally be included for purposes of creating an appropriate base composition or matrix suitable for topical administration including balms, creams, ointments, gels, salves, and the like. A typical base composition or matrix suitable for this aspect may be, but is not limited to, any one of several LUBRIDERM(r) skin moisturizing lotions (Johnson & Johnson). Such base materials can comprise from about 50% to about 85% (v/v) of a composition of the invention.

The composition optionally may also include one or more fragrance agent(s). Any type of natural or synthetic fragrance, such as floral, herbal or fruity fragrance could be utilized in accordance with the present invention. The use of fragrance is well known in the cosmetic arts and over-the-counter drug formulations, and many suitable fragrances are known.

Fragrance can comprise up to about 0.5% of the composition. Preferably, a fragrance comprises from about 0.05% to about 0.25% of the composition. An optimum concentration of fragrance is about 0.1% of the composition.

A composition of the present invention for topical application is preferably made in the form of an ointment, balm, cream, gel, salve, or the like, for application to an area of the body to prevent, reduce, or eliminate pain. For this purpose, the composition may contain one or more pharmaceutically acceptable topical carrier(s) which possess substantially non-irritating compatible components, either alone or in mixtures, suitable for topically delivering the active components. Thus, a composition of the invention may contain surfactants, solvents, including aqueous and non-aqueous solvents, fatty bodies, thickening agents, emulsifiers, or other pharmaceutically acceptable excipients, known to the skilled artisan which do not significantly alter the therapeutic effect of the active ingredient(s).

A composition of the present invention for topical administration may be packaged in any suitable container and/or dosage form including, for example, jars, squeeze tubes, suppositories, pads, wipes, or in a transdermal patch device infused with the composition.

### Dosage Forms

Pharmaceutical compositions of the present invention can be packaged and/or administered in various forms suitable for oral or topical (including transdermal, buccal, and sublingual) administration. Preferably, a composition according to the present invention is for topical administration.

Compositions for topical application can be formulated using methods known to the skilled artisan. In one embodiment, active agent(s) are mixed and combined with appropriate base substances in a ratio of about 1:3 or 1:4 to arrive at a suitably formulated composition that can be conveniently applied to the skin for topical administration to prevent, relieve, or eliminate pain. The base substance provides an appropriate vehicle for producing a cream, ointment, gel, salve, etc.

The compositions of the instant invention can comprise a wide range of components including components to assist in formulating for oral and/or topical administration, as is known in the art. The “CTFA Cosmetic Ingredient Handbook”, Second Edition, 1992, which is incorporated by reference herein in its entirety, describes a wide variety of cosmetic and pharmaceutical ingredients commonly used in the skin care industry, which are suitable for use in certain embodiments of the compositions of the instant invention. Reference also can be made to U.S. Pat. Nos. 6,013,271; 6,267,985; 4,992,478; 5,645,854; 5,811,111; and 5,851,543 herein incorporated by reference. Examples of such components include absorbents, anti-caking agents, antifoaming agents, antimicrobial agents, antioxidants, binders, buffering agents, such as sodium hydroxide, sodium citrate and EDTA, bulking agents, chelating agents, denaturants, dispersants, lubricants, external analgesics, moisturizers, thickeners, such as carboxymethylcellulose, opacifying agents, plasticizers, preservatives such as dichlorobenzyl alcohol and benzoic acid, reducing agents, skin-conditioning agents, suspending agents (nonsurfactant), gelling agents, such as petrolatum and mineral wax, ultraviolet light absorbers, and viscosity increasing agents (aqueous and nonaqueous).

In addition, a topical carrier composition may include a penetration enhancer defined as a material that increases the permeability of the skin to one or more active agents such as dimethylsulfoxide, dimethyl formamide, dimethylacetamide, decylmethylsulfoxide and polyethylene glycols.

Various lipids may be included in a topical preparation according to the present invention. As is known in the art, oils may be derived from animals, plants, nuts, petroleum etc. Those derived from animals, plant seeds and nuts are similar to fats and consequently can contain one or a significant number of more polar acids and/or ester groups. Oils derived from petroleum are usually aliphatic or aromatic hydrocarbons that are essentially free of polar substitution and therefore may be preferred for certain applications. It is preferable for the oil to be refined so as to be compatible with human tissue.

Other oil-based products that can be used include hydrocarbons or mineral fats obtained by the distillation of petroleum (petroleum jelly); vegetable oils and liquid triglycerides; animal fats or solid natural triglycerides; and waxes or solid ethers of fatty acids and organic alcohols. Lanolin or wool fats that are obtained from sheep wool and made up of fatty acids and cholesterol esters; and cetyl and stearyl alcohols, which are solid alcohols obtained by hydrogenation of their respective acids may also be used. Amphophilic compounds such as soaps or salts of fatty acids, that may be acidic or basic depending on whether the lipophilic group is anionic or cationic, sulfated alcohols which are semi-synthetic substances and synthetic surface active agents are known in the art and can be used in a topical composition of the invention.

Other materials that may be used in a topical preparation of the invention include liquid alcohols, liquid glycols, liquid polyalkylene glycols, liquid esters, liquid amides, liquid protein hydrosylates, liquid alkylated protein hydrosylates, liquid lanolin and lanolin derivatives, and other like materials. Particular examples include monohydric and polyhydric alcohols, e.g., ethanol, isopropanol, glycerol, sorbitol, 2-methoxyethanol, diethylene glycol, ethylene glycol, hexylene glycol, mannitol, cetyl alcohol and propylene glycol; ethers, such as diethyl or dipropyl ether; polyethylene glycols and methoxypolyoxyethylenes; carbowaxes having molecular weights ranging from 200 to 20,000; polyoxyethylene glycerols; polyoxyethylene; sorbitols; and stearoyl diacetin.

A number of different emulsifiers or surfactants can also be included in a topical composition of the invention. Emulsifiers can be ionic or non-ionic. Surfactants that can be used include the betaines, sultaines and hydroxysultaines. Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, steryl bis-(2-hydroxypropyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, stearyl betaine, lauryl dimethyl sulfoethyl betaine, lauryl bis-(2-hydroxyethyl) sulfopropyl betaine, and amidobetaines and amidosulfobetaines, oleyl betaine, and cocamidopropyl betaine). Examples of sultaines and hydroxysultaines include cocamidopropyl hydroxysultaine. Examples of other amphoteric surfactants are alkyliminoacetates, iminodialkanoates and aminoalkanoates.

Examples of anionic surfactants include the alkoyl isethionates, and the alkyl and alkyl ether sulfates, such as, ammonium cocoyl isethionate, sodium cocoyl isethionate, sodium lauroyl isethionate, sodium stearoyl isethionate and mixtures thereof, the sarcosinates, such as sodium lauroyl sarcosinate, sodium cocoyl sarcosinate, and ammonium lauroyl sarcosinate, sodium lauryl sulfate, ammonium lauryl sulfate, ammonium cetyl sulfate, sodium cetyl sulfate, sodium stearyl sulfate, ammonium cocoyl isethionate, Aerosol OT, sodium lauroyl isethionate, sodium lauroyl sarcosinate, castile soap, and mixtures thereof.

In another embodiment, compositions according to the invention are formulated for oral administration, preferably solid forms. Such formulation is within the ability of one skilled in the art, including the selection of pharmaceutically acceptable excipients and other ingredients. Compositions of the present invention for oral administration are generally prepared by methods known in the pharmaceutical formulation art (See e.g. Remington's Pharmaceutical Sciences, 18th. Ed., Mack Publishing Company, Easton, Pa., 1990).

The manner of formulating a composition of the present invention is conventional. Compositions for oral administration may he formulated as tablets, capsules, or solutions by including active agent(s) with one or more suitable binders, carriers, buffers, and the like, as is well-known to the skilled artisan.

For example, capsules are prepared by mixing one or more anti-oxidant preferably with at least one anti-inflammatory compound(s) and a suitable diluent, and filling the proper amount of the mixture into capsules. Suitable diluents include inert powdered substances such as starches of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. In this aspect, oral formulations optionally may incorporate diluents, binders, lubricants and disintegrators as well as the active compound(s). Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

In another embodiment transdermal patches may be used for topical delivery of a composition of the present invention using any suitable patch delivery system known to the skilled artisan, for example, those, disclosed in U.S. Pat. No. 6,096,334, and US Patent Application 2004/0043062, herein incorporated by reference. Typically, a patch comprises a resinous composition in which the active compound(s) will dissolve, or partially dissolve, which is held in contact with the skin by a film which protects the composition. Other, more complicated patch compositions may also be used, for example, those having a membrane pierced with pores through which active ingredient(s) is/are transferred by osmotic action. In general, compositions for transdermal administration contain from about 0.5% to about 50% of the active compounds in total, depending on the desired doses and the type of composition to be used.

### Therapeutic Methods

The compositions of the present invention are useful for treating, preventing, delaying, or reducing pain or ache in an individual suffering from a disease, injury, trauma, post-surgery pain, over-exertion, or any other cause that is associated with chronic and/or acute pain in any part of the body. Compositions of the invention may be used for medical treatment in humans and for veterinary treatment in animals.

Compositions of the present invention may be administered to treat pain associated with a disease or condition including, but not limited to, arthritis, including osteoarthritis and rheumatoid arthritis, bursitis, lupus, tennis elbow, tendonitis, fibromyalgia, sciatica, neuropathic pain, lower back pain, sore muscles, muscle cramps, muscle spasms, hip, knee, ankle, neck, foot, elbow, wrist, finger, shoulder, hand pain, and shingles.

Compositions of the present invention may also be administered to treat pain due to injury, trauma, or other condition associated, with acute and/or chronic pain. For example, a composition of the invention can be administered to treat pain associated with traumatic accident or injury, sports injury, strains, sprains, musculoskeletal injury or disease, post-surgery pain, insect stings, insect bites, and other like conditions that cause, or are associated with pain.

As used herein, “administer” or “administering” means to introduce or provide, such as to provide to a subject in need thereof a composition of the invention either by topical or oral administration. In a preferred embodiment the term refers to topical delivery or application, for example, by application of a cream, balm, or the like, directly on the surface of the skin, or by transdermal delivery using a patch-delivery device. Alternatively, a composition can be administered orally in the form of a tablet or capsule, or any other suitable dosage form for oral administration, as would be known to the skilled artisan.

As used herein, “treat” or “treating” or “treatment” can refer to one or more of preventing, delaying the onset of, eliminating, or reducing the severity of pain associated with a disease, disorder, injury, trauma, or condition e.g. arthritis and post-surgery pain. Also included is relief from other conditions that may be associated with pain, e.g. muscle and/or joint stiffness.

It has been discovered that compositions of the present invention are effective in treating pain. This is particularly advantageous in patients who are unable to secure relief from other means including, for example, treatment with aspirin or NSAIDs. In many instances, such patients develop allergies, or are precluded by other medical conditions from use of such agents, e.g. blood-thinning properties associated with the use of such agents. In other cases, alternative available agents are either ineffective pain relievers, or are associated with undesirable side effects including the risk of abuse and/or tolerance.

The present inventor has discovered that compositions of the invention provide effective pain relief that is long-acting. Administration of a composition of the invention, for example, by topical administration of a balm or cream, or the like, generally results in a step-wise reduction in pain that once achieved remains for an extended period of time. That is, generally, initial application of a topical composition of the invention results in a reduction in pain that is further reduced by one or more additional applications. For example, maximal pain reduction may be achieved by a plurality of applications, for example, from 1 to about 10; alternatively, from 1 to about 5; alternatively, still from 1 to about 3.

In one embodiment of the present invention, a composition is administered one or more times per week, once every three to four days; once every two to three days; once per day; or on an as needed basis, by topical application to an area of the body that is experiencing pain, e.g. knee, elbow, back, hip, neck, wrist etc. Topical application may be administered by rubbing a composition of the invention, e.g. a cream, balm, ointment, gel, or the like, directly on the skin over a site or portion of the body that is painful. Alternatively, topical application of a composition may be provided by means of a patch device that is impregnated with a composition of the invention. Generally, topical application would be provided as frequently as needed. For example, a patient suffering from arthritic pain might apply a topical composition several times daily; or once every three or four hours; or once before going to bed; or as often as needed.

The topical compositions of the present invention are generally effective in reducing pain. Unlike other pain-relieving compositions, the compositions of the present invention generally do not have to be applied multiple times per day. In use, a patient applying a topical composition of the present invention will generally experience pain relief in a period of about 2 hours to about 8 hours after application; alternatively between about 4 hours to about 6 hours after application. Significantly, the analgesic properties of a composition of the present invention generally remain effective for an extended period of time from about 24 hours to about 48 hours alter application.

The amount of active compound(s) in a composition of the present invention is best defined as the “effective amount”, that is, the amount of compound that provides the desired affect, i.e. reduction and/or elimination of pain in a patient in need of such treatment.

Dosages of a composition of the present invention to be administered to a patient must, in the final analysis, be set by a physician or by the patient on an ad hoc basis. General outlines of dosages for specific compounds and/or components are provided hereinbelow.

The following discussion describes ranges and concentrations for compositions according to specific embodiment(s) of the present invention including preferred embodiment(s). However, it is to be understood that the present invention is not limited to such embodiment(s), and other embodiments based on the disclosure presented herein will be readily apparent to one of ordinary skill in the art.

In one embodiment of the present invention, a composition contains omega-3 and/or omega-6 fatty acids, or a source of such fatty acids, which include EPA, DHA, and gamma linolenic acid (GLA) as anti-inflammatory agents. These components can be provided by any suitable source, for example, Fish oil, Borage oil, Evening Primrose oil, Flaxseed oil, and Black Current oil.

In one embodiment, the ratio of EPA and DHA to GLA in a composition of the invention is from about 2 to about 0.5; preferably the ratio is about 0.9.

In a preferred embodiment of the present invention, a composition of the invention comprises one or more additional anti-inflammatory agents including Bromelain, White Willow bark extract, and Boswellia. These components provide anti-inflammatory activity that enhances the ability to treat and/or reduce pain associated with trauma, injury, or disease(s) such as, for example, arthritis and/or other joint swelling diseases.

Bromelain can comprise from about 1% to about 5% by weight of the composition. Preferably, Bromelain comprises from about 3% to about 4% of the composition. White Willow bark extract can comprise from about 5% to about 15% by weight of the composition. Preferably, White Willow bark extract comprises about 10% of the composition. Boswellia can comprise from about 5% to about 15% by weight of the composition. Preferably, Boswellia comprises about 12% of the composition.

In a preferred embodiment of the present invention, a composition further comprises Cat's Claw. The active component(s) in Cat's-Claw is/are believed to possess immune system boosting activity. Cat's Claw may also possess anti-inflammatory activity.

Cat's Claw can comprise from about 5% to about 15% by weight of the composition. Preferably, Cat's Claw comprises from about 10% to about 12% of the composition.

A composition of the invention preferably comprises one or more agent(s) that are naturally derived agents in the form of raw substances such as barks, extracts, powders, pastes, distillates, teas, oils, etc. Alternatively, compositions of the invention may comprise purified active ingredients. For example, the anti-oxidant pycnogenol can be derived from French Maritime Pine Bark extract. Alternatively, the active ingredient(s) in pycnogenol, believed to be one or more proanthocyanidins, can be provided as purified substances. A composition of the invention may include any/or all such sources for active agents.

For oral administration, a composition according to one embodiment can be in the form of one or more tablets, capsules, soft gels, or gel caps containing the components in the approximate dosages listed in Table 1.

TABLE 1

| Component | Daily Dosage |
|---|---|
| Pine Bark extract | 8-60 mg |
| Boswellia Extract | 80-240 mg |
| White Willow Bark extract | 80-240 mg |
| Cat's Claw | 40-200 mg |
| Fish Oil | 40-200 mg |
| Bromelain Powder | 20-60 mg |
| Borage Oil | 400-1200 mg |

A composition for oral administration may additionally contain fillers and other excipients suitable form formulating as tablets, capsules, softgels, or gelcaps, as would be known to the skilled artisan.

The following examples are provided as illustrations of some embodiments of the invention and are not intended to limit the scope of the claimed subject matter in any way.

### EXAMPLES

### Example 1

### Formulation for Topical Application

A topical skin cream composition according to one embodiment of the invention is shown in Table 2. The activity of each component and their proportions are provided in units of percent by weight. Also included in Table 2 is a listing of ingredients in each component including active ingredients, in the gravimetric and percent by weight in each component.

TABLE 2
Cream for topical administration

| | | Component | | Ingredient |
|---|---|---|---|---|
| Activity | Component | Percentage | Ingredient | Percentage |
| Anti-oxidant | 0.5 g French | 1.4 | 0.45 g | 3.7 |
| | Maritime Pine | | Proanthocyanidins | |
| | Bark extract | | | |
| | (Pycnogenol) | | | |
| Anti- | 4.3 g | 12.2 | 2.79 g Boswellic | 23.1 |
| inflammatory | Boswellia | | acids | |
| | extract | | | |
| NA | 4.3 g Aerosol | 12.2 | 3.23 g Dioctyl | 26.3 |
| | OT 75% | | sodium | |
| | | | sulfosuccinate | |
| Anti- | 3.2 g White | 9.1 | 0.48 g salicin | 4.0 |
| inflammatory | willow bark | | | |
| | extract | | | |
| Immune | 4.0 g Cats | 11.4 | NA | <0.8 |
| system | Claw | | | |
| NA | 2.0 g Lecithin | 5.7 | | |
| Anti- | 3.7 g Fish oil | 10.5 | 1.33 g EPA | 11.1 |
| inflammatory | | | 0.99 g DHA | 8.2 |
| Anti- | 1.2 g | 3.4 | 0.1 g Bromelain | 0.8 |
| inflammatory | Bromelain Powder | | | |
| Anti- | 12.0 g Borage | 34.1 | 2.65 g gamma | 22.0 |
| inflammatory | oil | | linolenic acid | |

NA—not known or not applicable

### Example 2

### Preparation of Cream Formulation for Topical Application

The following procedure was followed to prepare a topical composition according to the present invention.

### Preparation of Aqueous Phase (Part A)

In a clean container, add and mix:
10 Tablets Pycnogenol (Broken)¹ Note 1—each tablet of pycnogenol contains 50 mg French Maritime Pine Bark extract.
14 Tablets Boswellia Extract² Note 2—each tablet contains 307 mg Boswellia Extract.
25.0 g deionized water

To the mixture was added:
43 g Aerosol OT

Stir and then add:
7.5 g White Willow Extract (obtained from 16 Caps White Willow Standardized Extract)

The Part A ingredients were blended and occasionally stirred for about 2 hours.

### Preparation of Non-Aqueous Phase (Part B)

In a clean container, add and mix:
4.0 g Cats Claw
2.0 g Lecithin Granules
1.2 g Bromelain powder
4.0 g Omega-Max Liquid
12.0 g Borage Oil

Part B ingredients were blended and stirred occasionally for about 1 hour. Then Part A was added to Part B and mixed well. The mixture was filtered and 24.3 g of the filtrate was blended with 72.9 g of LUBRIDERM® lotion to produce a cream. The final formulation was stored overnight at 4° C. prior to use.

### Example 3

### Topical Pain Relief

The cream formulation of Example 1 was provided to a 78 year old male patient suffering from pain and stiffness in both hips and knees. The patient could not take NSAIDs. The cream was topically applied in the morning to all affected areas. By mid-afternoon the pain in all four affected areas had substantially subsided, and the patient reported no pain the next day.

### Example 4

### Topical Pain Relief

The formulation of Example 1 is provided to a 60-year old female with osteoarthritis having pain and stiffness in the lower back and hands. On day 1, she applies a single topical application of the cream to each of the painful areas. The next day she reports reduced and/or eliminated pain in all areas to which the cream was applied.

### Example 5

### Topical Formulation 2

### Part A:

5 pycnogenol tablets crushed and mixed with 5 ml water;
Contents of 8 capsules Boswellia;
Contents of 8 capsules White willow extract;
1.0 g Lecithin granules;
0.6 g Bromelain;
2.0 g Cats claw;
2.0 g Omega max liquid;
6.0 g Borage oil.

### Part B

4.6 g Docusate

The ingredients of Part A were mixed in a clean vessel and combined with the Docusate (Part B) in another vessel. The residue left in vessel 1 was rinsed with 5 grams isoproply alcohol and added to the combined ingredients in vessel 2. The blended ingredients were stored overnight at room temperature and then filtered. To 5 g of the filtrate was added 15 g of Lubriderm to produce a cream formulation.

### Example 6

### Topical Formulation 3

| | Comments |
|---|---|
| A. Aqueous Phase Ingredients | |
| 10 tablets Pycnogenol | Each tablet contains 50 mg French |
| | maritime Pine Bark extract; rich source of |
| | anti-oxidant proanthocyanidins |
| 14 tablets Boswellia extract | Each tablet contains 307 mg Boswellia |
| | extract (standardized to 65% Boswellic |
| | Acids; 200 mg Oleo-resin-gum) |
| 4.3 g Aerosol OT, 75% | |
| 20 g deionized water | |
| B. Non-aqueous Phase | |
| Ingredients | |
| 7.6 g White Willow | 16 capsules: each capsule contains about |
| Standardized Extract | 250 mg White Willow bark; 200 mg |
| | White Willow extract; 15% salicin |
| 4.0 g Cat's Claw Herbal | |
| Powder | |
| 2.0 g Lecithin granules | |
| 4.0 g Omega-Max Liquid | 5 ml contains: |
| | 4.62 g Marine fish oil concentrate; 1.66 g |
| | Eicosapentaenoic acid; 1.25 g |
| | Docosahexaenoic acid; Vitamin E; lemon |
| | oil; rosemary extract |
| 1.2 g Bromelain powder | Each gram contains: 86.5 mg Bromelain; |
| | 3000 MCU/gram; 2000 GDU/gram |
| 12.0 g Borage Oil, 22% | |
| GLA content | |

The aqueous ingredients were combined and stirred about 3 hours at room temperature to achieve dissolution. The non-aqueous ingredients were combined and stirred occasionally over a 3 hour period. Thereafter, the aqueous and non-aqueous mixtures were combined and stirred over a 2 hour period. The combined mixture was stored overnight at 4° C. and then filtered. To about 12.8 g of filtrate was added about 38.5 g LUBRIDERM® lotion resulting in a soft cream.

Example 7

### Treatment of Patient with Post-Operative Knee Pain

The formulation of Example 1 was provided to a 45-year old female with post-operative pain in one knee which was especially severe at night. The patient reported that the pain was so severe that she was frequently awakened several times during the night. Her previous treatment prior to presentation had involved a period of 6 months of applying a prescription gel product she received from her orthopedic physician. While the gel provided some relief the patient reported continued sleep disruption due to knee pain. When she applied the formulation of Example 1 by rubbing the composition on the post-operative knee just prior to bedtime, she was able to sleep through the night without pain. When she arose in the morning, she reported being able to walk down the hall without limping. She experienced similar relief after a second night of treatment, and has continued regular use of the formulation with no further nighttime sleep disruption due to pain.

I claim:

1. A pharmaceutical composition for the treatment of pain comprising:
a) an anti-inflammatory agent selected from the group consisting of aloe vera, MSM, emu oil, chondroitin, glucosamine, a capsaicinoid, arnica extract, grape seed extract, coriander oil, marigold extract, nettle leaf extract Roman chamomile oil, blue-bottle extract, St. John's wort, White willow bark extract, witch hazel extract, feverfew extract, barley grass, black cohosh, black snakeroot, bugbane, squawroot Boswellia, plant-based oil, borage oil, bromelain, burdock, calendula, cayenne, dandelion, devil's claw root, DHEA (dehydroepiandosterone), Echinacea, EFAs (essential fatty acids such as omega-3 and omega-6 fatty acids including linoleic acid (LA) and alpha linolenic acid (LNA)), fish oil, green tea, pomegranate extract, ginger root, hyssop, elderflower, evening primrose oil, flaxseed, ginkgo, ginger, ginseng, Hawthorne, kaempferol, licorice, life root, golden Senecio, squaw weed, golden groundsel, cocash weed, coughweed, ragwort, golden ragwort, linden, marjoram, meadow sweet NDGA, neem, neem oil, Padma 28, quercetin, turmeric, wild yam, wormwood, yucca and combinations thereof;
b) an anti-oxidant agent selected from the group consisting of chondroitin, vitamin C, grape seed extract, St. John's wort extract, coriander oil, barley grass, bilberry. Echinacea, garlic, ginger, ginkgo, ginseng, grape seed proanthocyanidin extract (GSPE), green tea, Hawthorne, lemon balm, milk thistle, oregano, peppermint, pomegranate juice, purslane, pycnogenol, French Maritime Pine Bark extract, red wine, rosemary, schizandra, wuweizi, wurenchun, trilinolein, sanchi, turmeric, d-alpha Tocopherol, β-carotene, selenium, and vitamin C ester, and combinations thereof; and
c) one or more agents selected from the group consisting of buffer, surfactant, thickening agent, vitamin, emulsifier, preservative, solvent, bulking agent, topical base composition, moisturizer, humectant gelling agent, and essential oil.

2. A pharmaceutical composition as in claim 1 wherein said anti-inflammatory agent is selected from Boswellia extract, White Willow bark extract, Fish oil, Bromelain, plant-based oil, and Borage oil and said anti-oxidant is selected from pycnogenol, French Maritime Pine Bark extract, GSPE, pomegranate juice, and green tea, and further comprising an immune system boosting agent.

3. A pharmaceutical composition as in claim 2 for topical application for the treatment of pain, wherein said anti-inflammatory agent is Boswellia extract in a range of about. 3% to about 15% of said composition; White Willow Bark extract in a range of about 5% to about 15% of said composition; fish oil containing EPA and DHA in a range of about 4% to about 15% of said composition; and plant-based oil containing GLA in a range of about 20% to about 60% of said composition; and wherein said anti-oxidant is French Maritime Pine Bark extract in a range of about 1% to about 8% of said composition; and wherein said immune system boosting agent is Cat's Claw in a range of about 2% to about 20% of said composition; and wherein said one or more agents is a surfactant in a range of about 2% to about 10% and an emulsifier in a range of about 1% to about 10% of said composition.

4. The composition of claim 3 wherein the surfactant is selected from Aerosol OT and castile soap and said emulsifier is lecithin.

5. A composition as in claim 4 wherein said White Willow Bark extract has a salicin content range of about 10% to about 30% by weight.

6. A pharmaceutical composition as in claim 5 wherein said plant-based oil has a GLA content of about 7% to about 25% by weight.

7. A pharmaceutical composition as in claim 6 wherein said French Maritime Pine Bark extract has a proanthocyanidin content of about 60% to about 100% by weight.

8. A pharmaceutical composition for topical application for the treatment of pain comprising by weight:
a. about 12% Boswellia extract,
b. about 14% White Willow Bark extract;
c. about 10% fish oil wherein said fish oil contains EPA and DHA;
d. about 6% French Maritime Pine Bark extract; and
e. about 45% plant-based oil, wherein said plant-based, oil contains GLA.

9. A pharmaceutical composition for topical application for the treatment of pain comprising by weight:
a. about 22% EPA and DPA;
b. about 4% salicin;
c. about 24% boswellic acid;
d. about 45% gamma linolenic acid; and
e. about 5% proanthocyanidins; and
f. about 50% to about 90% (v/v) skin moisturizing lotion.

10. A method for treating pain in a mammal comprising administration of an effective amount of a composition of claim 1.

11. A method as in claim 10 wherein said composition is in a form selected from cream, ointment, and balm, and wherein said administration is by topical application.

12. A method as in claim 10 wherein said pain is chronic or acute, and is associated with a condition selected from the group consisting of disease, trauma, injury, over-exertion, or post-surgery.

13. A method as in claim 12 wherein said condition is a musculoskeletal or neurological disease selected from the group consisting of osteoarthritis, degenerative arthritis, rheumatoid arthritis, fibromyalgia, lupus, neuropathy, and sciatica.

14. A method for treating pain in a patient in need thereof by topical application of a composition of claim 3.

15. A method as in claim 14 wherein said application is repeated at least once resulting in a stepwise reduction in pain with each application.
